(19) United States
(12) Patent Application Publication
(10) Pub. No.: US 2010/0233192 A1
(43) Pub. Date: Sep. 16, 2010

(54) Manufacturing Method of Activated Lymphocytes for Immunotherapy

(75) Inventors: Soon Won Park, Busan (KR); Young Ok Son, Busan (KR); Cheol Hun Son, Busan (KR); You Soo Park, Busan (KR); Jung Hwa Ban, Busan (KR); Kyoung-Gyu Lee, Busan (KR); Jeong Su Jang, Busan (KR); Chi Dug Kang, Busan (KR); Won-Suk Kim, Busan (KR); Kyung Chool An, Busan (KR); Back Chun Lee, Busan (KR); Ju In Kim, Busan (KR); Eun Kyung Park, Busan (KR); Sung Hee Choi, Busan (KR)

Correspondence Address: CLARK & ELBING LLP, 101 FEDERAL STREET, BOSTON, MA, 02110, US

(73) Assignee: Binex Co., Ltd., Busan (KR)

(21) Appl. No.: 12/438,148

(22) PCT Filed: Apr. 18, 2007
(86) PCT No.: PCT/KR2007/001893

§ 371 Date: Apr. 17, 2009

(30) Foreign Application Priority Data
Aug. 23, 2006 (KR) 10-2006-0079705

(51) Int. Cl. A61K 39/00; C12N 5/078; A61P 37/04

(52) U.S. Cl. 424/184.1; 435/377; 435/404

(57) ABSTRACT

Disclosed is a method for preparing activated lymphocytes, which comprises isolating lymphocytes from peripheral blood and proliferating and activating the isolated lymphocytes in vitro. According to the disclosed method, highly effective toxic cells can be prepared in large amounts by culturing human peripheral lymphocytes in the presence of an anti-CD3 antibody, IFN-γ and IL-2. The activated lymphocytes proliferated according to the disclosed preparation method comprise both CD3-CD56+ (natural killer cell marker) cells that are the main components of LAK cells, and CD3+CD56+ cells that are the main components of CIK cells, and can be cultured in large amounts. Thus, the lymphocyte cells can show a significantly higher anticancer effect compared to when the LAK cells and the CIK cells are used alone.

### TECHNICAL FIELD

The present invention relates to a method for preparing activated lymphocytes, and more particularly to a method for preparing activated lymphocytes, which can be used as cellular immunotherapeutic agents either by isolating lymphocytes from human peripheral blood, proliferating and activating the isolated lymphocytes in large amounts in vitro and administering the activated lymphocytes to the person from which the lymphocytes originated, or by cryopreserving the activated lymphocytes, and administering the cryopreserved lymphocytes to the person from which the lymphocytes originated, when the person has a disease against which the administration of the immune cells is required.

### BACKGROUND ART

Human immune cells include natural killer (NK) cells and T lymphocytes, which can recognize and eliminate transformed cells such as cancer cells or virus-infected cells. Thus, the use of function of such cells will have preventive and therapeutic effects against these diseases. However, in the case of cancer patients, it is difficult for immune cells to show sufficient anticancer effects, because the immune system is weakened due to various anticancer therapies, including surgery, anticancer drug therapy and radiation therapy, so as to weaken the function of immune cells or to significantly reduce the number of immune cells. For this reason, if immune cells from a patient are proliferated and activated in vitro in large amounts, and then administered to the autologous patient, it is possible to expect high anticancer effects. An activated lymphocyte is a cellular immunotherapy product for treating cancer by proliferating and activating human blood immune cells in vitro in large amounts and administering the activated immune cells to the autologous person, and it is an individually tailored anticancer immunotherapeutic agent for inducing in vivo immune by activating the immune cells of the autologous patient, like an anticancer immunotherapeutic agent comprising dendritic cells.

Since tumor antigens were known, it became possible to specifically eliminate tumor cells using cytotoxic T lymphocytes (CTL) [Rosenberg et al., 1999]. However, it is known that the expression of MHC class I in various carcinomas is reduced, and due to this mechanism, cancer cells can evade immune surveillance by CTL [Amiot et al., 1998]. On the other hand, On the other hand, MHC class I-deficient cancer cells are highly sensitive to natural killer (NK) cells [Pawelec et al., 2004].

NK cells can eliminate cancer cells and virus-infected cells without recognizing antigens [Albertsson et al., 2003; Colucci et al., 2003; Smyth et al., 2002]. The activity of NK cells is regulated by a balance of signals from activating signals and inhibitory signals [Farag et al., 2003]. The most well activating ligands are NKG2D ligands. Among them, the expression of MHC class I-related chain A and B (MICA/B) is induced by stresses such as heat shock, oxidative stress and viral infection, and the expression of UL-16 binding proteins (ULBPs) is induced by viral infection [Vivier et al., 2002]. The expression of the NKG2D ligands is induced by stresses, and these ligands show various expression patterns in various cancer cell lines [Watzl et al., 2003].

That the NKG2D ligands have the capability to label stressed or transformed cells means that the sensitivity of cancer cells to NK cells can be controlled by the regulation of expression of activating ligands. Because the NKG2D ligands can increase sensitivity to NK cells, cancer cells with a high expression of NKG2D ligands can be eliminated, even though the expression of MHC class I is normal [Raulet et al., 2003]. Thus, if the expression of NKG2D ligands can be increased, the anticancer therapeutic effects of NKG2D receptor-expressing cells such as NK, NKT, CD8+T and γδ T cells can be further increased.

In the beginning of 1980s, the Rosenberg group performed clinical tests of LAK cells (lymphokine-activated killer cells; NK cells activated by IL-2) on melanoma, renal cell carcinoma, lymphoma, lung cancer and colon cancer [Rosenberg et al., 1985], but the use of the LAK cells in clinical applications was limited, because the anticancer cytotoxicity of the LAK cells was relatively weak and it was difficult to secure the cells in large amounts. In an attempt to overcome this limitation, Schmidt-Wolf et al. developed technology of preparing highly effective toxic cells in large amounts by culturing peripheral blood monocytes in the presence of an anti-CD3 antibody, IFN-γ, IL-1 and IL-2 [Schmidt-Wolf et al., 1991]. The cultured cells are called “CIK (cytokine-induced killer) cells”, which have high cytotoxicity and proliferation rate compared to the existing LAK cells. It is known that, in this cell group, cells showing toxicity effects are cells positive for the CD56 surface antigen, about 20-30% of which are CD3+CD56+ cells, and the ratio of CD3-CD56+ cells (NK cell surface antigens) is very low (<10%). However, studies on the cytotoxicity of cells comprising CIK cells against tumor cells revealed that the killing ability of CD3-CD56+ cells was higher than that of CD3+CD56+ cells [Schmidt-Wolf et al., 1993; Lu et al., 1994; Scheffold et al., 1995]. Therefore, in LAK cells and CIK cells, cells having the highest anticancer effect can be considered to be NK cells (CD3-CD56+).

Meanwhile, MS Dilber et al. developed technology of culturing CD3-CD56+ in large amounts by culturing peripheral blood monocytes in an anti-CD3 antibody and IL-2 [Carlens et al., 2001]. The cultured cells are called “CINK (cytokine-induced natural killer) cells”, which have high proliferation rate compared to the existing LAK cells. However, according to this method, CD3-CD56+ cells could be cultured in large amounts only in CellGro SCGM media supplemented with an anti-CD3 antibody and IL-2.

Also, it is known that CD4+CD25+ regulatory T cells are present in peripheral blood monocytes (PBMCs) of normal persons at a ratio of less than 5% and suppress the proliferation of T cells in vitro [K. E. Earle et al., 2005]. Furthermore, CIK cells cultured with IL-2 in vitro can induce the proliferation of CD4+CD25+ regulatory T cells that secrete a large amount of IL-10, thus suppressing the proliferation of CTL and reducing the cytotoxicity of CTL [Jan Schmidt et al., 2004]. Particularly, it is known that CD4+CD25+ regulatory T cells reduce the amount of an NKG2D receptor that is expressed in CIK or NK cells, and TGF-β produced by activated CD4+CD25+ regulatory T cells inhibits the cytotoxicity of NK cells [Francois G et al., 2005]. Accordingly, in the present invention, the ratio of CD4+CD25+ regulatory T cells in autologous activated lymphocytes differentiated and proliferated from PBMC is remarkably reduced to 5%, thus improving the in vivo function of the autologous activated lymphocytes.

### DISCLOSURE OF INVENTION

Accordingly, the present inventors have made many efforts to solve the problems occurring in the prior art and, as a result, have found that CD56+ and NKG2D+ cells having excellent killing ability against tumor cells and virus-infected cells can be prepared in large amounts by culturing lymphocytes isolated from human peripheral blood, in the presence of interleukin-2 (IL-2), interferon-gamma (IFN-γ) and an anti-CD3 antibody, thereby completing the present invention.

It is therefore a main object of the present invention to provide a method of preparing activated lymphocytes, in which CD56+ and NKG2D+ cells having excellent killing ability against tumor cells and virus-infected cells can be prepared in large amount by culturing lymphocytes isolated from human peripheral blood, in the presence of an anti-CD3 antibody, IL-2 and IFN-γ.

Another object of the present invention is to provide a cellular immunotherapeutic composition comprising, as active ingredients, activated lymphocytes proliferated according to said method.

### BRIEF DESCRIPTION OF THE DRAWINGS

FIG. 1 shows measurement results for the number of activated lymphocytes at 6 days, 10 days, 15 days and 21 days of culture.

FIG. 2 shows graphs obtained by analyzing surface antigens CD3 and CD56 in activated lymphocytes of each test group using flowcytometry.

FIG. 3 shows graphs obtained by analyzing surface antigens NKG2D and CD56 in activated lymphocytes of each test group using flowcytometry.

FIG. 4 shows graphs obtained by analyzing surface antigens CD16 and CD56 in activated lymphocytes of each test group using flowcytometry.

FIG. 5 shows graphs obtained by analyzing surface antigens CD3 and CD56, surface antigens NKG2D and CD56 and surface antigens CD16 and CD56 in activated lymphocytes using flowcytometry before and after freezing the activated lymphocytes.

FIG. 6 shows the CD4- and CD25-positive cells in the CIK (cytokine-induced killer) cells of normal persons, cultured in a medium supplemented with an anti-CD3 antibody and IL-2.

FIG. 7 shows results obtained by analyzing surface antigens CD4 and CD25 in activated lymphocytes using flowcytometry at 0 days, 14 days and 21 days of culture.

### BEST MODE FOR CARRYING OUT THE INVENTION

To achieve the above objects, according to one aspect of the present invention, there is provided a method for preparing activated lymphocytes, comprising the steps of: (1) collecting and isolating lymphocytes from peripheral blood; (2) culturing the lymphocytes in vitro in the presence of interleukin-2 (IL-2), interferon-gamma (IFN-γ) and an anti-CD3 antibody to prepare activated lymphocytes; (3) cryopreservating the activated lymphocytes for a given period of time; and (4) thawing and restoring the lymphocytes of the cryopreservation step. The steps (3) and (4) are used when the long-term storage of the activated lymphocytes prepared through the steps (1) and (2) is required.

The step (1) of the method of the present invention is a step of collecting and isolating lymphocytes from peripheral blood, in which the lymphocytes are collected from the peripheral blood of a person with disease or a healthy person. The collection of blood from arm veins is preferred because it is convenient and easy, but any material can be used as long as it contains lymphocytes. The amount of peripheral blood collected is preferably about 0.001-500 ml, and more preferably about 10-100 ml.

Heparin, EDTA or citric acid may be added to the peripheral blood collected in the step (1), such that the coagulation of the blood does not occur. The activated lymphocytes of the present invention can be obtained by isolating lymphocytes from the collected peripheral blood and proliferating and activating the isolated lymphocytes through in vitro culture.

The step (2) of the method according to the present invention is a culture step of activating and proliferating the lymphocytes isolated in the step (1). The method of culturing the lymphocytes collected in the step (1) is not specifically limited, but is preferably carried out in the presence of IL-2, IFN-γ or an anti-CD3 antibody alone, or combinations thereof. In this case, it is most preferable to culture the lymphocytes in the presence of a combination of IL-2, IFN-γ and an anti-CD3 antibody, because this can show an excellent anticancer effect compared to the case of culturing the lymphocytes in the presence of IL-2, IFN-γ or an anti-CD3 antibody alone. Moreover, antigen-specific activated lymphocytes can also be obtained by inducing antigen-specific T lymphocytes using a suitable antigen and then adding a CD3 antibody, a CD3 antibody or various mitogen thereto. As the antigen, it is possible to use a purified antigen, an extract from cancer cells or viruses, a cancer cell or virus itself, or a pseudo-antigen having cross-reactivity therewith, and in this case, any material can be used as long as it has a function of proliferating and activating the lymphocytes. Meanwhile, IL-15 may also be used instead of said IL-2 in the culture process.

IL-2 and IFN-γ, which are used in the step (2) of the inventive method, are commercially available and are preferably used in a concentration of 1-2000 U/ml in the culture medium. Also, IL-2 and IFN-γ can be used after they are dissolved in generally widely used cell culture media, for example, physiological saline, phosphate buffer solution, RPMI-1640, DMEM, IMDM, AIM-V (GIBGO, USA), X-Vivo (Cambrex), LGM, KBM-306 (KohjinBio), CellGro (CellGenix), etc. Once IL-2 and IFN-γ are dissolved, they need to be stored in a cold or frozen state in order to prevent the activity thereof from decreasing. Meanwhile, as the culture medium, any medium can be used without any particular limitation as long as it is suitable for the culture of lymphocytes, and preferred examples thereof may include RPMI-1640, DMEM, IMDM, AIM-V, X-Vivo, LGM, KBM and CellGro, particularly preferred being serum-free media, such as AIM-V, CellGro, KGM and X-Vivo.

The culture medium, which is used in the step (2), preferably contains serum, because the serum-containing medium has an excellent proliferation effect.

As the serum, not only commercially available bovine fetal serum or normal person's serum, but also autologous serum, may be used. Also, it is possible to use a serum-free medium. The culture of the lymphocytes can be carried out in a general cell culture system, for example, a CO₂ incubator. The concentration of CO₂ in the cell culture is in the range of 1-10%, and preferably in the range of 3-7%, and the culture temperature is in the range of 30-40° C., and preferably in the range of 35-38° C.

Although the period of cell culture is not specifically limited, the cell culture is preferably carried out for about 2-28 days because it is secured that the stimulus information of the anti-CD3 antibody is transferred to the cells. A culture period of 3-8 days is particularly preferable because it enables the stimulus information to be stably transferred to the cells and shows high culture efficiency. It is more preferable to observe the state of the cells with a microscope during the culture period to measure the number of the cells while adding a suitable amount of culture medium. Also, in the cell culture, the cells do not proliferate 1-4 days after the start of culture, but the proliferation of the cells is observed after that, and when the cells start to normally proliferate, the culture medium changes from orange to yellow. The amount of additional medium added is preferably about 0.1-5 times the amount of the culture medium to which the additional medium is to be added. Meanwhile, the addition of the additional medium is performed at an interval of 1-7 days, and preferably 2-4 days, in order to prevent the deterioration of the culture medium and the reduction of IL-2 activity.

The cell culture in the step (2) of the inventive method can be initiated by suspending monocyte cells in a culture medium, containing IL-2 and IFN-γ, and adding the anti-CD3 antibody to a culture container for immobilization. Furthermore, when various cytokines and mitogens, if necessary, are added to the culture medium, the efficiency of proliferation and activation of the lymphocytes will be further increased. Also, the anti-CD3 antibody, which is used for the stimulation of the lymphocyte cells, may be an antibody produced and purified from animals or cells, or a commercially available OKT-3 antibody. In addition to these antibodies, any antibody may be used without any particular limitation as long as it can stimulate the proliferation and activation of the lymphocytes. For example, an anti-CD28 antibody may also be used.

The step (3) of the inventive method is a step of cryopreserving the activated lymphocytes for a given period of time. In this step, the lymphocytes to be preserved may be suspended in a cell preservative solution at a concentration suitably selected depending on the size thereof. It is required to suspend and cryopreserve the lymphocytes in the preservative solution at a density of 1×10³ cells/ml to 1×10¹⁰ cells/ml. Although the amount of the cell preservative solution used in this step is not important, the preservative solution is preferably used in the range of 0.1-1000 ml in view of convenience, and more preferably in the range of 0.5-100 ml.

The cryspreservative solution for use in the step (3) of the inventive method may be a commercially available cell preservative solution or can be self-prepared for use in the step (3). The cell preservative solution may contain serum, polymer substances such as proteins or polysaccharides, and dimethyl sulfoxide (hereinafter, referred to as “DMSO”) in a suitable buffer solution or basal medium, and all the listed substances are not required for the cells to be preserved. Thus, a preservative solution enabling cell preservation has no limitation on the composition thereof. The lymphocytes are suspended in a suitable cell preservative solution and cryopreserved at low temperatures. The prepared cryopreservative solution may be stored in a refrigerator (4° C.) after preparation until use.

In the inventive method for the preparation of activated lymphocytes, the activated lymphocytes are preferably CD56+ and NKG2D+ cells. In the present invention, the CD56+ is a killer cell marker, and the NKG2dD+ is a lymphocyte-activating receptor marker.

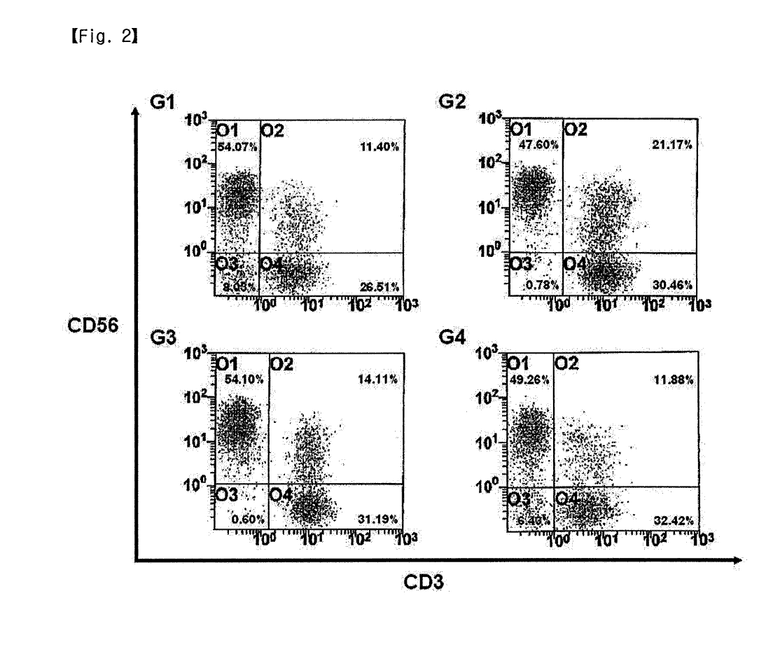

The x-axis of graphs in FIG. 2 showing the results of Example 3 of the present invention is for lymphocytes labeled with CD3 (T-lymphocyte marker), and the y-axis is for lymphocytes labeled with CD56. Each of the graphs is divided into four sections for analysis, and among the four sections, the left upper section (region O1) indicates CD3-negative and CD56-positive natural killer cells (NK cells), and the right upper section (region O2) indicates CD3- and CD56-positive lymphocytes. The cells in these two sections (regions O1 and O2) are lymphocytes laving anticancer effects. In such analysis results, when lymphocytes were cultured in the presence of a combination of IL-2, IFN-γ and an anti-CD3 antibody according to the embodiment of the present invention, the expression ratio of killer cell surface antigen CD56 was higher than 60% in all test groups (G1 to G4). Also, the ratio of T lymphocyte marker CD3-negative cells was higher than 50% in all the test groups (G1-G4). Such results suggest that AIM-V, CellGro, X-Vivo and KBM media used in the present invention are all useful for the mass culture of killer cells having excellent anticancer effects.

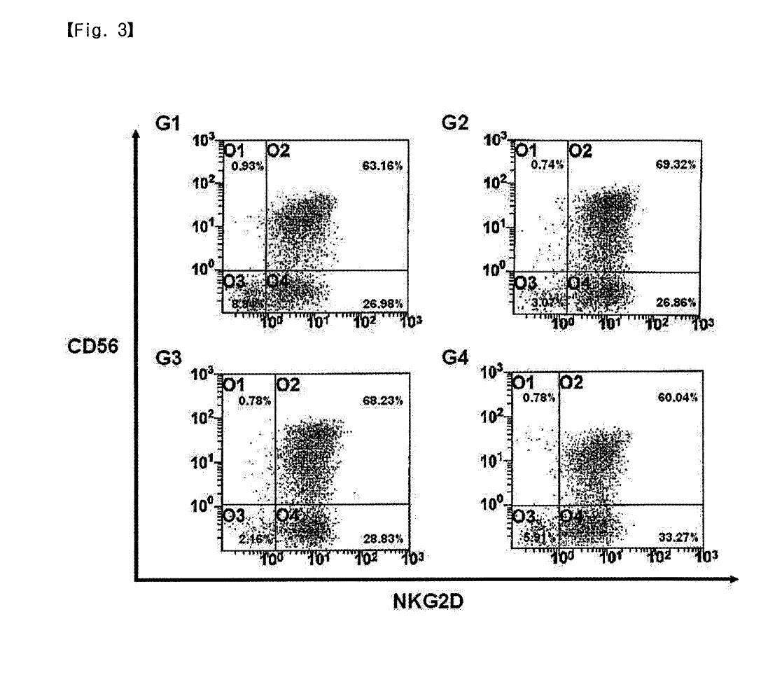

Meanwhile, FIG. 3 shows analysis results for the expression of an NKG2D receptor that is one of activating receptors known to be involved in the activation of lymphocytes, such as NK, NKT, CD8T and γδ T cells. The x-axis of graphs in FIG. 3 is for lymphocytes labeled with NKG2D, and the y-axis is for lymphocytes labeled with CD56. Each graph was divided into four sections for analysis, and among the four sections, the left upper section (region O2) and the right lower section (region O4) indicate NKG2D-positive lymphocytes. Particularly, the right upper section indicates lymphocytes positive for both NKG2D and CD56. Thus, when lymphocytes were cultured in the presence of a combination of IL-2, IFN-γ and an anti-CD3 antibody according to the embodiment of the present invention, the ratio of NKG2D-positive lymphocytes in all test groups (G1-G4) was higher than 90%, and NKG2D was positive in all most all killer cells (CD56-positive cells. From such results, it can be seen that activated lymphocytes cultured according to the preparation method of the present invention show high activity against tumor cells or virus-infected cells.

In the inventive preparation for preparing activated lymphocytes, the activated lymphocytes preferably further comprises, in addition to CD56+ and NKG2D+, CD16+. In the present invention, said CD16+ is an Fc gamma RIII marker.

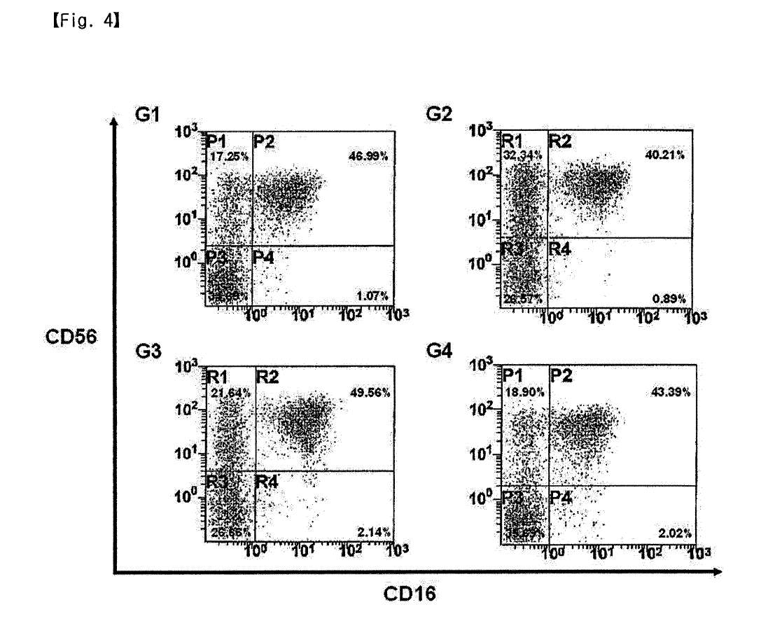

The x-axis of graphs in FIG. 4 showing the results of Example 3 is for lymphocytes labeled with CD16, and the y-axis is for lymphocytes labeled with CD56. Each graph was divided into four sections for analysis, and among the four sections, the right upper section (region P2) indicates lymphocytes positive for both CD16 and CD56. The CD16 surface antigen is known to induce antibody-dependent cell-mediated cytotoxocity (ADCC). Thus, it is considered that, because the ratio of surface antigen-expressing cells in cells prepared according to the embodiment of the present invention was higher than 40% in all test groups, the cells prepared according to the present invention would show more potent anticancer effects compared to CIK cells which are known to express little or no CD16 surface antigen.

In the inventive method for preparing activated lymphocytes, the ratio of CD4+ and CD25+ in the activated lymphocytes is preferably 3-6%, and more preferably less than 5%.

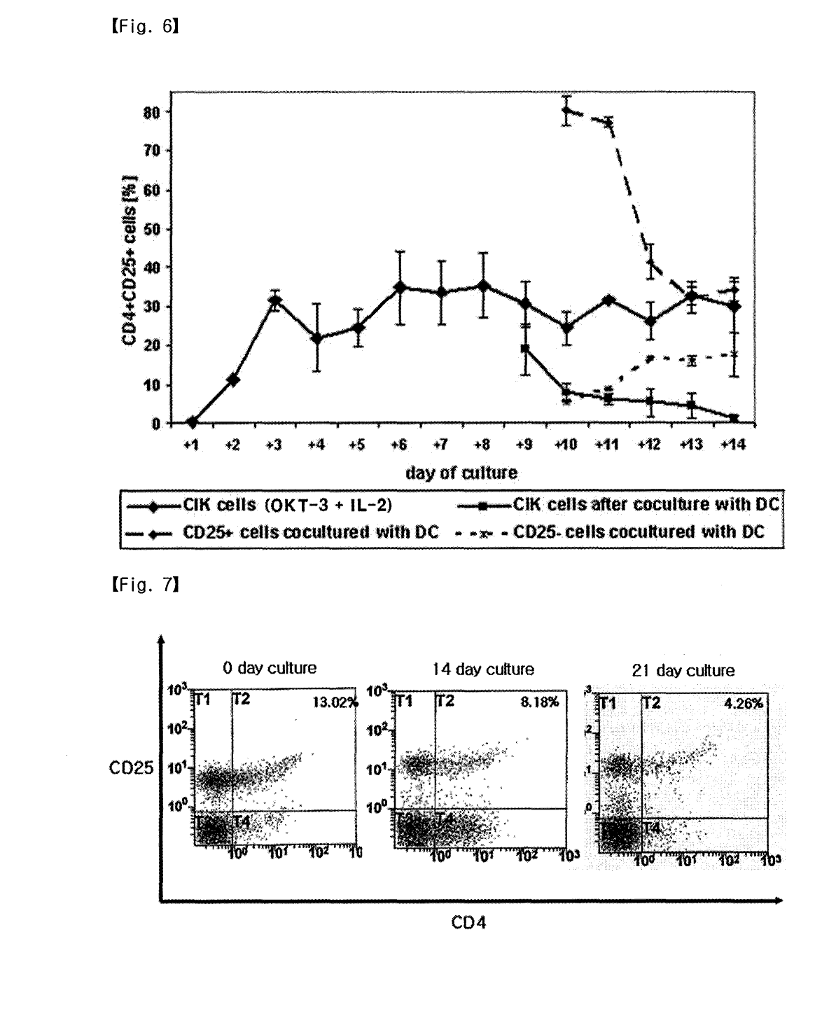

It is known that CD4- and CD25-positive cells in the peripheral blood of cancer patients are 2.5 times larger than those in normal persons [Anna Maria Wolf et al, 2003]. Also, the CD4- and CD25-positive cells are known to increase in CIK (cytokine-induced killer) cells cultured from the peripheral blood of normal persons. That is, it was reported that, even in the case of the CIK cells of normal persons, when these cells were cultured in the presence of the anti-CD3 antibody (or OKT-3) and IL-2, the ratio of CD4- and CD25-positive cells was increased from 0.5±0.07% before culture to 35.5±8.4% after 14 days of culture (see FIG. 6) [Jan Schmit et al., 2004]. In the present invention, the ratio of CD4- and CD25-positive cells in lymphocytes isolated from the peripheral blood of cancer patients was shown to be more than 10%, but it was reduced to a normal level of less than 5%, when the lymphocytes were cultured in the presence of IFN-γ, an anti-CD3 antibody and IL-2 for 21 days (see FIG. 7).

In the inventive method for preparing activated lymphocytes, the anti-CD3 antibody is preferably immobilized to a culture container before use.

For use in the present invention, the anti-CD3 antibody is preferably contained in a culture medium, but it is more preferably immobilized to culture container in view of lymphocyte-proliferation efficiency and operation easy. Culture container for immobilizing the antibody may include culture containers made of glass, polyurethane, polyolefin or polystyrene. Specifically, an easily available cell culture flask made of plastic can be used and the size thereof can be suitably selected. The immobilization of the antibody can be performed by adding a dilution of the anti-CD3 antibody to the culture container for immobilization and standing the antibody, for example, at 4-37° C. for 2-24 hours. Also, for the immobilization of the anti-CD3 antibody, the anti-CD3 antibody is diluted in a physiological buffer saline such as sterilized phosphate buffer at a concentration of 0.1-30 μg/ml. The antibody can be stored in a refrigerator (4° C.) after immobilization until use. For use in the present invention, the liquid component is removed from the stored antibody dilution, and the remaining antibody may, if necessary, be washed with physiological buffer solution such as phosphate buffer solution at room temperature.

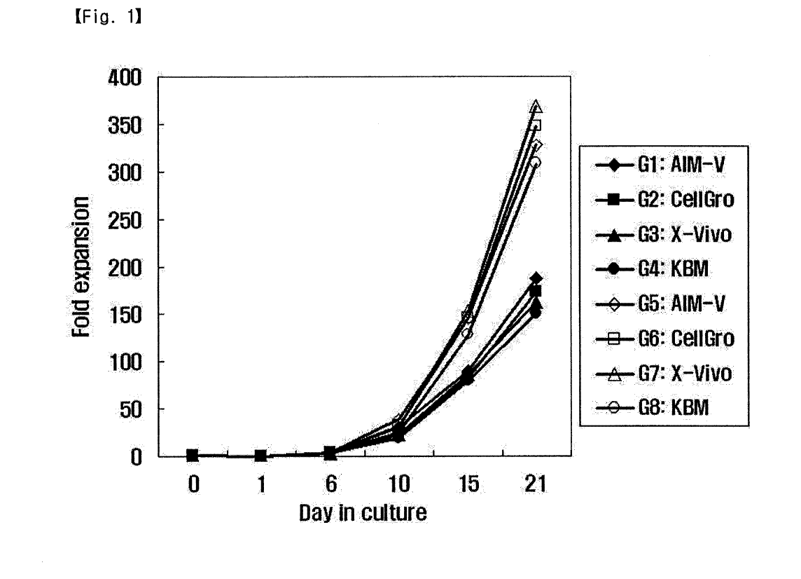

FIG. 1 shows measurement results for the number of activated lymphocytes at 6 days, 10 days, 15 days and 21 days of culture. In FIG. 1, lymphocytes were cultured in AIM-V medium for G1 and G5, CellGro medium for G2 and G6, X-Vivo medium for G3 and G7, and KBM medium for G4 and G8. Measurement results for the number of activated lymphocytes at 21 days of culture showed that, when lymphocytes were cultured in a medium containing an anti-CD3 antibody (G1-G4), the number of activated lymphocytes increased by 168 times in average as compared to the number of lymphocytes at an early stage of culture, and when lymphocytes were cultured in an anti-CD3 antibody-immobilized flask (G5-G8), the number of activated lymphocytes increased by 338 times in average. As can be seen from the above results, when lymphocytes were cultured in the anti-CD3 antibody-immobilized flask, the proliferation rate of the cells was about two times higher than the case of culture in the anti-CD3 antibody-containing medium AIM-V, and the mass culture of activated lymphocytes was possible in all CellGro, X-Vivo and KBM media.

Also, the proliferation and activation of cells were compared between the case of performing culture using the anti-CD3 antibody-immobilized flask and the case of performing culture using the medium containing the anti-CD3 antibody. As a result, when cells were cultured in the anti-CD3 antibody-immobilized flask, the proliferation rate of the cells was about two times higher than the other case. Also, the analysis of surface antigens CD3, CD16, CD56 and NKG2D at 21 days of culture showed that there was no difference according to culture conditions.

In the inventive method of preparing activated lymphocytes, the cryspreservation of the cells is preferably performed using a freezing tube or bag at a cell density of 0.5-10.0×10⁷ cells/freezing tube or 0.05-10.0×10¹⁰ cells/freezing bag.

A freezing container, in which the frozen cells of the present invention are to be preserved, may be a commercially available freezing cell freezing tube or bag, and the size thereof can be suitably selected. The number of the cells to be frozen is preferably 0.5-10.0×10⁷ cells/freezing tube, and the number of freezing tubes is in the range of 2-1000 depending on the amount of blood collected. In the case of freezing bags, the number of the cells to be frozen is preferably 0.05-10.0×10¹⁰ cells/freezing bag, and the number of freezing bags is in the range of 1-10 depending on the amount of blood collected. Also, for use in the present invention, the frozen cells are thawed, lysed and restored to the patient. In a special case, the cells may also be administered immediately after thawing and lysis.

In the inventive method for preparing activated lymphocytes, the cells are preserved for a maximum of 15 years, can be thawed, lysed and restored at a suitable point of time, if necessary, and can be cryopreserved for a long period of time. The cryopreserved cells may be stored according to any cell cryopreservation method known to one skilled in the art, but the cells can be stored for 15 years or longer, when the freezing tube or bag containing the cells is cooled to −70 to −90° C. at a rate of −1° C./min using a controlled rate freezing system, and then transferred to and stored in a nitrogen tank.

The cryopreservation of the present invention can be performed using a freezer, an ultra-low-temperature freezer or a nitrogen tank, but it is preferable to use a controlled rate freezing system in view of the stability and proliferation efficiency of lymphocytes. As the controlled rate freezing system, a commercially available system can be used and a system developed by a user may also be used. Moreover, the frozen cells are stored in the controlled rate freezing system for 0-30 days, and preferably 0-7 days. As used herein, the term “0 days” means that the period of cryopreservation in the controlled rate freezing system is 1-24 hours. As used herein, the term “period” refers to a preliminary freezing period just before the activated lymphocytes are transferred to a nitrogen tank capable of storing the cells for a long period of time. The transfer of the cells from the controlled rate freezing system to the nitrogen tank may also be performed using any commercially available article, which can enter the nitrogen tank, including a can or a freezing tube box.

According to another aspect of the present invention, there is provided a medium composition for the culture of activated lymphocytes, the composition comprising an anti-CD3 antibody, interleukin-2 (IL-2) and interferon-gamma (IFN-γ). The prior technology of culturing CD3-CD56+ cells can be performed only in a CellGro SCGM medium supplemented with the anti-CD3 antibody and IL-2, but in the present invention, large amounts of CD3-CD56+ cells can be cultured not only in CellGro SCGM medium, but also in AIM-V, CellGro DC, KBM-306 and X-Vivo media, in the presence of IL-2, the anti-CD3 antibody and IFN-γ.

According to still another aspect of the present invention, there is provided a cellular immunotherapeutic composition comprising, as active ingredients, activated lymphocytes proliferated according to the preparation method of the present invention.

As used herein, the cellular immunotherapeutic agent is an anticancer immunotherapeutic agent for treating cancer by proliferating and activating human blood immune cells in large amounts in vitro and administering the activated cells to an autologous patient. Also, it is an individually tailored anticancer therapeutic agent for inducing in vivo immune by activating the autologous immune cells of a patient, like an anticancer immunotherapeutic agent comprising dendritic cells.

In the present invention, when the peripheral blood lymphocytes of not only normal persons, but also terminal cancer patients, were cultured in the presence of an anti-CD3 antibody, IFN-γ and IL-2, activated lymphocytes having substantially the same activity could proliferate in large amounts.

Also, when the activated lymphocytes obtained according to the preparation method of the present invention were thawed and restored after they were cryopreserved for a long period of time, the viability and activity of the cells were maintained. Thus, the activated lymphocytes according to the present invention can be used as cellular immunotherapeutic agents either by isolating lymphocytes from the peripheral blood of a patient with disease or a healthy person, proliferating and activating the isolated lymphocytes in vitro and administering the activated lymphocytes to the autologous patient, or by cryopreserving the activated lymphocytes, and thawing and restoring the cryopreserved cells, when the person from which the lymphocytes originated has a disease against which the administration of the immune cells is required.

The cellular immunotherapeutic composition of the present invention can be prepared in the form of general formulations known in the art, for example, an injectable solution, and can be surgically transplanted directly into a cancer site or can migrate into a cancer site after intravenous administration. Although the dose of the composition according to the present invention can vary depending on the type of disease, the route of administration, the age and sex of the patient, and the severity of the disease, the inventive composition is preferably administered at a dose of 1×10⁷-10¹¹ cells for average adults.

According to the present invention, highly effective toxic cells can be prepared in large amounts by culturing human peripheral blood lymphocytes in the presence of an anti-CD3 antibody, IFN-γ and IL-2. The activated lymphocytes proliferated according to the preparation method of the present invention comprise both CD3-CD56+ (natural killer cell marker) cells that are the main component of LAK cells, and CD3+CD56+ cells that are the main components of CIK cells, and can be cultured in large amounts. Thus, the activated lymphocytes according to the present invention can show significantly higher anticancer effects compared to when LAK cells and CIK cell cells are used alone.

### EXAMPLES

Hereinafter, the present invention will be described in further detail with reference to examples. It is to be understood, however, that these examples are intended to merely illustrate the present invention and are not to be construed to limit the scope of the present invention.

### Example 1

### Blood Collection and Isolation of Lymphocytes

10-100 ml of peripheral blood was collected from human veins in an aseptic state. As the blood collection container, a blood collection tube or bag containing an anticoagulant such as heparin or EDTA was used. Then, the blood was injected into a 50-ml centrifugal tube and mixed well with the same amount of phosphate buffer saline (PBS). Histopaque-1077 solution (Sigma) was added to the 50-ml centrifugal tube such that the ratio of Histopaque-1077 to the PBS-diluted blood was 1:2 to 1:4. Then, the PBS-diluted blood was added slowly to the centrifugal tube such that the liquid surface was not scattered. Then, the mixture was centrifuged in conditions of revolution of 400×g and room temperature, and the lymphocyte fraction was isolated. Then, the fraction was washed three times with a suitable amount of phosphate buffer saline. After the last centrifugal washing, the supernatant was removed, the lymphocyte precipitate was well suspended in phosphate buffer saline, and the number of the lymphocytes was measured using a trypan blue solution. As a result, the total cell number was 2.0×10⁷ to 2.0×10⁸.

### Example 2

### Preparation of Anti-CD3 Antibody-Immobilized Flask

10 ml of an anti-CD3 antibody solution (Orthoclone OKT3 injection manufactured by Ortho Biothech) prepared by adding the antibody to phosphate buffer saline at a concentration of 5 μg/ml was added to a culture flask having a bottom area of 225 cm² and was allowed to spread uniformly on the bottom surface. The next day, the antibody solution in the flask was sucked with a suction pump and washed three times with phosphate buffer saline, thus preparing an anti-CD3 antibody-immobilized flask.

### Example 3

### Culture of Activated Lymphocytes

In the present invention, the proliferation rate and activation of activated lymphocytes were compared between different culture conditions. For this purpose, a suspension of the lymphocytes was added to and mixed well with 50 ml of each of suitable media (G1: AIM-V (GIBGO, USA); G2: CellGro (CellGenix); G3: KBM (Kohjin Bio); and G4: X-Vivo (Cambrex)), each containing 1000 U/ml IFN-γ (Leucogen, LG Life Sciences) and 1-5% human serum. Then, each of the media was cultured in a cell culture flask in condition of 37° C. and 5% CO₂. After 24 hours of culture, the culture medium in each flask was collected and transferred to a fresh 225-cm² T-flask, and 500 U/ml IL-2 (Proleukin, CHIRON) and 50 ng/ml anti-CD3 antibody (Orthoclone, Ortho Biotech) were added to each of the flasks. Meanwhile, the proliferation and activation of cells were compared between the case of performing culture using the anti-CD3 antibody-immobilized flask prepared in Example 2 and the case of performing culture using a medium containing the anti-CD3 antibody. After 5 days, the culture medium in each of the flasks was collected, and transferred to a 225-cm² T-flask. Then, 50 ml of IL-2-containing culture medium (hereinafter, referred to as “culture medium”) was added to each flask and cultured at 37° C. in the presence of 5% CO₂. After 4 days, 100 ml of culture medium was added to each flask and cultured at 37° C. in the presence of 5% CO₂. During the culture period, 500 U/ml of interleukin-2 was added at an interval of 2-3 days. At 14-15 days of culture, the number of flasks was increased in order to prevent the overcrowding of activated lymphocytes, and the cells were cultured at 37° C. in the presence of 5% CO₂ for 21 days, thus obtaining 5.0×10⁸-5.0×10¹⁰ activated lymphocytes.

FIGS. 2 to 4 show results obtained by analyzing surface antigens in activated lymphocytes using flowcytometry at 21 days of culture. Specifically, FIG. 2 shows analysis results for surface antigens CD3 and CD56, FIG. 3 shows analysis results for surface antigens NKG2D and CD56, and FIG. 4 shows analysis results for surface antigens CD16 and CD56. In FIGS. 2 to 4, lymphocytes was cultured in AIM-V medium for G1, CellGro medium for G2, X-Vivo medium for G3, and KBM medium for G4. Also, surface antigens CD3 and CD56, surface antigens NKG2D and CD56 and surface antigens CD16 and CD56 in activated lymphocytes of each test group were analyzed at 21 days of culture using the anti-CD3 antibody-immobilized flask. As a result, the expression of each of the surface antigens was almost similar to the case of performing the culture of lymphocytes in a medium containing the anti-CD3 antibody.

In the process of preparing activated lymphocytes according to the preparation method of the present invention, even when cryopreserved peripheral blood lymphocytes were cultured in the presence of the anti-CD3 antibody, IL-2 and IFN-γ, the proliferation and activation of the lymphocytes well occurred.

### Example 4

### Ratio of Immune-Suppressing T Cell CD4+/CD25+ in Activated Lymphocytes

In the present invention, the expression of CD4+/CD25+ cells that are immune-suppressing T cells, which induce immune tolerance, was analyzed using flowcytometry at various points of time during culture. FIG. 7 shows results obtained by analyzing surface antigens CD4 and CD25 in activated lymphocytes using flowcytometry at 0 days, 14 days and 21 days of culture. As can be seen in FIG. 7, the expression level of CD4 and CD25 started to decrease with the passage of culture time, and was reduced to a normal level (less than 5%) after 21 days of culture.

### Example 5

### Cryopreservation of Activated Lymphocytes

The 21-day-cultured activated lymphocytes of each test group, obtained in the step (3), were collected and centrifuged. Then, each of the culture media was removed to obtain an activated lymphocyte precipitate. The activated lymphocyte precipitate was mixed well with a cell preservative solution (medium 199 containing 7-15% DMSO, 0.1-10% penta-starch, 0.1-10% heparin and 1-20% albumin), and 1.0 ml of the lymphocyte solution was dispensed into each of 10 cell preservative tubes (Corning) according to culture conditions. Then, the cell preservative tubes were cooled to −90° C. at a rate of −1° C./min using a controlled rate freezing system, and transferred to and stored in a nitrogen tank.

### Example 6

### Thawing and Analysis of Cryopreserved Activated Lymphocytes

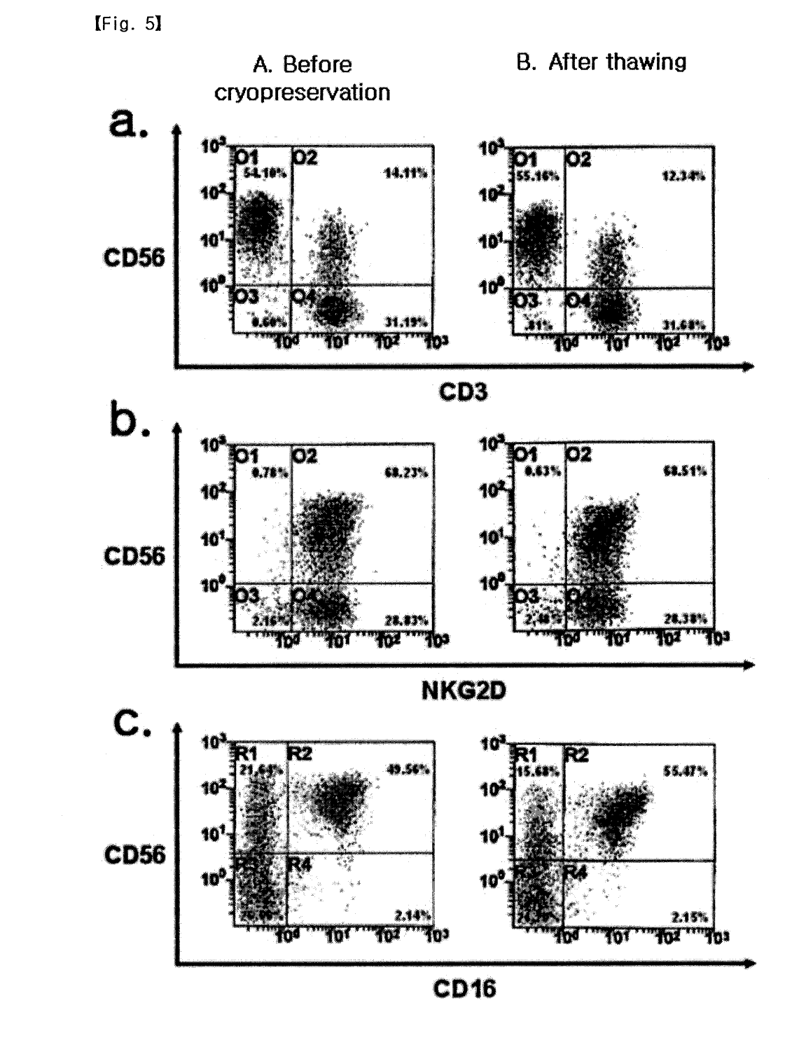

After 60 days of storage, among the tubes cryopreserved in the step (4), three tubes per each of the test groups were taken, thawed in a constant-temperature water bath for 1-4 minutes, washed three times with media to remove the cell preservative solution, and suspended in the culture media. Then, the cells were measured for viability using a trypan blue solution, and the measurement results showed that the cell viability was in the range of 70-80% depending on culture conditions (see Table 1). Meanwhile, each of suspensions of the activated lymphocytes cultured in various conditions as described above was cultured in a T75-cm² T-flask at 37° C. in the presence of 5% CO² for 2-3 days, and then the viability of the activated lymphocytes was measured. The measurement results showed that the cell viability was higher than 95%. Also, analysis results for surface antigens CD3, CD16, CD56 and NKG2D in the lymphocytes showed that the ratio of each surface antigen in the lymphocytes was almost similar between before and after the cryopreservation of the lymphocytes. From such results, it is considered that the long-term cryopreservation of the activated lymphocytes had no significant effect on the activity of the activated lymphocytes (see FIG. 5).

TABLE 1
Cell viabilities of various test groups
before and after cell cryopreservation

| | | Before | After | Cell | |
|---|---|---|---|---|---|
| Group | Tube No. | cryopreservation | thawing | viability | Average |
| G1 | G1-1 | 5.0 | 4.1 | 82 | 78.7 |
| | G1-2 | 5.0 | 3.8 | 76 | |
| | G1-3 | 5.0 | 3.9 | 78 | |
| G2 | G2-1 | 5.0 | 4.2 | 84 | 81.3 |
| | G2-2 | 5.0 | 3.9 | 78 | |
| | G2-3 | 5.0 | 4.1 | 82 | |
| G3 | G3-1 | 5.0 | 4.0 | 80 | 76.7 |
| | G3-2 | 5.0 | 3.9 | 78 | |
| | G3-3 | 5.0 | 3.6 | 72 | |
| G4 | G4-1 | 5.0 | 4.1 | 82 | 78.7 |
| | G4-2 | 5.0 | 3.8 | 76 | |
| | G4-3 | 5.0 | 3.9 | 78 | |

### INDUSTRIAL APPLICABILITY

In the present invention, in order to overcome the limitation of the prior methods for preparing activated lymphocytes, there is provided a preparation method by which CD56+ and NKG2D+ cells having excellent killing ability against tumor cells and virus-infected cells can be cultured in large amounts by culturing lymphocytes, isolated from human peripheral blood, in the presence of an anti-CD 3 antibody, IL-2 and IFN-γ. Thus, the activated lymphocytes proliferated and activated according to the inventive method can be used as cellular immunotherapeutic agents to greatly increase anticancer effects. Also, according to the present invention, activated lymphocytes obtained by proliferating and activating the peripheral blood lymphocytes of a healthy person can be frozen and preserved for a long period time, and some time later, when the person from which the activated lymphocytes originated has a disease against which the administration of immune cells is required, the preserved activated lymphocytes can be used as cellular immunotherapeutic agents to treat the disease.

1. A method for preparing activated lymphocytes, comprising the steps of:
collecting and isolating lymphocytes from peripheral blood;
culturing the lymphocytes in vitro in the presence of interleukin-2 (IL-2), interferon-gamma (IFN-γ) and an anti-CD3 antibody to prepare activated lymphocytes;
cryopreserving the activated lymphocytes for a given period of time; and
thawing and restoring the lymphocytes of the cryopreservation step.

2. The method of claim 1, wherein the activated lymphocytes are CD56+ and NKG2D+.

3. The method of claim 2, wherein the activated lymphocytes further include, in addition to CD56+ and NKG2D+, CD16+.

4. The method of claim 1, wherein the ratio of CD4+ and CD25+ in the activated lymphocytes is 3-6%.

5. The method of claim 1, wherein anti-CD3 antibody is immobilized to a culture container before use.

6. The method of claim 1, wherein the cryopreservation is performed using a freezing tube or bag at a cell density of 0.5−10.0×10⁷ cells/freezing tube or 0.05-10.0×10¹⁰ cells/freezing bag.

7. A medium composition for the culture of activated lymphocytes, the composition comprising an anti-CD3 antibody, interleukin-2 (IL-2) and interferon-gamma (IFN-γ).

8. A cellular immunotherapeutic composition comprising, as active ingredients, activated lymphocytes proliferated using the preparation method of claim 1.
